(19) United States
(12) Patent Application Publication
(10) Pub. No.: US 2026/0110674 A1
(43) Pub. Date: Apr. 23, 2026

(54) NANOCOMPOSITE FILMS FOR VOC SENSING AND METHODS OF USING SAME

(71) Applicant: Georgia Tech Research Corporation, Atlanta, GA (US)

(72) Inventor: Jie Xu, Atlanta, GA (US)

(21) Appl. No.: 19/116,992

(22) PCT Filed: Sep. 29, 2023
(86) PCT No.: PCT/US2023/075479

§ 371 Date: Mar. 28, 2025

Related U.S. Application Data
(60) Provisional application No. 63/377,750, filed on Sep. 30, 2022.

(51) Int. Cl. G01N 33/00; G01N 27/12

(52) U.S. Cl. CPC G01N 33/0047; G01N 27/127

(57) ABSTRACT

An exemplary embodiment of the present disclosure provides a system for detecting the presence of an analyte. The system can comprise a first electrode, a second electrode, and a nanocomposite material. The nanocomposite material can comprise a polymer material and a plurality of conductive nanoparticles dispersed in the polymer material. The nanocomposite material can be in electrical communication with the first and second electrodes.

### CROSS-REFERENCE TO RELATED APPLICATIONS

This application claims the benefit of U.S. Provisional Application Ser. No. 63/377,760, filed on 30 Sep. 2022, which is incorporated herein by reference in its entirety as if fully set forth below.

### FIELD OF THE DISCLOSURE

The various embodiments of the present disclosure relate generally to sensors, and more particularly, to sensors for volatile organic compounds.

### BACKGROUND

Gas sensors can be found in diverse applications including indoor/outdoor environmental monitoring, pollution control, public safety, health monitoring, and food and agriculture. Gas chromatography (GC) coupled with mass spectrometry (MS) is the gold standard to detect a wide range of volatile organic compounds (VOC). However, GC/MS can require bulky, expensive, power and time consuming equipment and trained personnel. Portable and low-cost gas sensors could be used in the field to provide real time monitoring on the levels of targeted gases.

A number of VOC sensors have been developed including chemoresistors, electrochemical based sensors, photo-ionization detectors (PID), micro GC (μGC). Among all the VOC sensors, chemoresistors are more compact, low cost, and easy to measure. The chemoresistors are a family of sensors that change electrical resistance in the presence of an analyte of interest. Diverse sensing materials including metal oxides, nanomaterials, conductive polymers (CPs) have been used for VOC sensing. However, metal oxide based chemoresistors require high temperature operation. CPs have the advantages of tunable electrical/electronic properties, easy synthesis, structural diversity, flexibility, and environmental stability. However, CPs have intrinsic limitations, such as a relatively low conductivity, low sensitivity and a lack of consistency in properties.

Chemoresistors based on organothiol-functionalized gold nanoparticle thin films have been reported. These types of chemoresistors change their resistances through the partitioning of analytes into the organothiol caps surrounding the nanoparticles. Although longer chain alkyl of thiol molecules is preferred for an increased detection sensitivity, the significant reduced conductivity associated with long chain thiol limits the length of chain that can be used as the capping molecule.

Accordingly, there is a need for improved sensors that overcome one or more of these disadvantages associated with conventional sensors.

### BRIEF SUMMARY

An exemplary embodiment of the present disclosure provides a system for detecting the presence of an analyte. The system can comprise a first electrode, a second electrode, and a nanocomposite material. The nanocomposite material can comprise a polymer material and a plurality of conductive nanoparticles dispersed in the polymer material. The nanocomposite material can be in electrical communication with the first and second electrodes.

In any of the embodiments disclosed herein, the system can be configured such that exposure of the analyte to the nanocomposite material alters an impedance between the first and second electrodes.

In any of the embodiments disclosed herein, the system can be configured such that exposure of the analyte to the nanocomposite material can cause the nanocomposite material to swell, altering a spacing of the plurality of conductive nanoparticles within the polymer material.

In any of the embodiments disclosed herein, the polymer material can have a first set of Hansen solubility parameters and the analyte can have a second set of Hansen solubility parameters, wherein a difference between the first set of Hansen solubility parameters and the second set of Hansen solubility parameters to obtain a less than 1 for interaction parameter χ₁₂ in the Flory-Huggins equation.

In any of the embodiments disclosed herein, the polymer material can have a first set of Hansen solubility parameters and the analyte can have a second set of Hansen solubility parameters, wherein a difference between the first set of Hansen solubility parameters and the second set of Hansen solubility parameters to obtain a less than 0.75 for interaction parameter χ₁₂ in Flory-Huggins equation.

In any of the embodiments disclosed herein, the polymer material can have a first set of Hansen solubility parameters and the analyte can have a second set of Hansen solubility parameters, wherein a difference between the first set of Hansen solubility parameters and the second set of Hansen solubility parameters to obtain a less than 0.5 for interaction parameter χ₁₂ in Flory-Huggins equation.

In any of the embodiments disclosed herein, the system can be configured to detect the presence of the analyte when the analyte is exposed to the polymer material at a concentration level of 50 parts per billion (ppb).

In any of the embodiments disclosed herein, the analyte can be either an inorganic gas or organic gas including volatile organic compound (VOC).

In any of the embodiments disclosed herein, the VOC can be selected from the group consisting of cyclododecanol, benzoic acid, 4-ethoxy-, ethyl ester, 3,4,4-trimethyl-3-pentanol, and combinations thereof.

In any of the embodiments disclosed herein, the plurality of conductive nanoparticles can be selected from the group consisting of gold nanoparticles, silver nanoparticles, and carbon nanoparticles.

In any of the embodiments disclosed herein, the plurality of conductive nanoparticles can be gold nanoparticles.

In any of the embodiments disclosed herein, the plurality of conductive nanoparticles can be homogeneously dispersed in the polymer material.

In any of the embodiments disclosed herein, the nanocomposite material can be in the form of a nanocomposite film.

In any of the embodiments disclosed herein, the plurality of nanoparticles can have an average particle size of less than 10 nm.

In any of the embodiments disclosed herein, the plurality of nanoparticles can have an average particle size of less than 5 nm.

Another embodiment of the present disclosure provides a method of detecting whether an analyte is present in a fluid. The method can comprise: exposing the fluid to a nanocomposite material, the nanocomposite material comprising a polymer material and a plurality of conductive nanoparticles dispersed in the polymer material; and measuring, after exposing the fluid to the nanocomposite material, an impedance of the nanocomposite material. The impedance can be indicative of whether the analyte is present in the fluid.

In any of the embodiments disclosed herein, exposing the fluid to the nanocomposite material can cause the nanocomposite material to swell, altering a spacing of the plurality of conductive nanoparticles within the polymer material.

In any of the embodiments disclosed herein, the method can be capable of detecting the presence of the analyte when the analyte is present in the fluid at a concentration level of 50 parts per billion (ppb).

These and other aspects of the present disclosure are described in the Detailed Description below and the accompanying drawings. Other aspects and features of embodiments will become apparent to those of ordinary skill in the art upon reviewing the following description of specific, exemplary embodiments in concert with the drawings. While features of the present disclosure may be discussed relative to certain embodiments and figures, all embodiments of the present disclosure can include one or more of the features discussed herein. Further, while one or more embodiments may be discussed as having certain advantageous features, one or more of such features may also be used with the various embodiments discussed herein. In similar fashion, while exemplary embodiments may be discussed below as device, system, or method embodiments, it is to be understood that such exemplary embodiments can be implemented in various devices, systems, and methods of the present disclosure.

### BRIEF DESCRIPTION OF THE DRAWINGS

The following detailed description of specific embodiments of the disclosure will be better understood when read in conjunction with the appended drawings. For the purpose of illustrating the disclosure, specific embodiments are shown in the drawings. It should be understood, however, that the disclosure is not limited to the precise arrangements and instrumentalities of the embodiments shown in the drawings.

FIG. 1 provides an illustration of a nanocomposite film before (left) and after (right) exposure to a sample containing targeted volatile organic compounds (VOC), in accordance with some embodiments of the present disclosure.

FIG. 2 provides a TEM picture of alkanethiol coated Au nanoparticles, in accordance with some embodiments of the present disclosure.

FIG. 3 provides a schematic of an experimental setup to measure the VOC using a nanocomposite coated sensing board, in accordance with some embodiments of the present disclosure.

FIG. 4 provides a plot of sensing data for a 0.5 ppm decanone collected at two different flow rates, using a sensing method in accordance with some embodiments of the present disclosure.

FIG. 5 provides a plot of sensing data for five consecutive VOC detections, using a sensing method in accordance with some embodiments of the present disclosure.

FIG. 6 provides a plot of sensing data for fluids containing 2 concentrations of 2-decanone, ethanol, and acetone, using a sensing method in accordance with some embodiments of the present disclosure.

FIG. 7 provides a flow chart of an exemplary method of sensing whether an analyte is present in a fluid, in accordance with some embodiments of the present disclosure.

FIG. 8 provides a diagram of a system for detecting the presence of an analyte, in accordance with some embodiments of the present disclosure.

FIG. 9 provides a graph that exemplifies the relationship between nanocomposite conductivity and filler volume fraction of nanoparticles contained within a polymer that forms a nanocomposite film, in accordance with some embodiments of the present disclosure.

FIG. 10 provides a graph illustrating the relationship between conductivity of a nanocomposite or film containing gold nanoparticles (GnP) with and without chloroauric acid (HAuCl4) in an acrylate polymer (PMMA) based on the volume fraction of gold nanoparticles present in the film, in accordance with some embodiments of the present disclosure.

### DETAILED DESCRIPTION

To facilitate an understanding of the principles and features of the present disclosure, various illustrative embodiments are explained below. The components, steps, and materials described hereinafter as making up various elements of the embodiments disclosed herein are intended to be illustrative and not restrictive. Many suitable components, steps, and materials that would perform the same or similar functions as the components, steps, and materials described herein are intended to be embraced within the scope of the disclosure. Such other components, steps, and materials not described herein can include, but are not limited to, similar components or steps that are developed after development of the embodiments disclosed herein.

The present disclosure provides sensors for detecting the presence of an analyte in a fluid and methods of using such sensors. As shown in FIG. 8, an exemplary embodiment of the present disclosure provides a system for detecting the presence of an analyte. The system can comprise a first electrode 205, a second electrode 210, and a nanocomposite material 215.

The first 205 and second 210 electrodes can be many different electrodes known in the art. The first 205 and second 210 electrodes can be made of many different conductive materials, including, but not limited to, metals, conductive polymers, carbon, and combinations thereof. Electrical connections (e.g., wires) can be connected to the electrodes 205 210 to deliver signals to a controller.

The nanocomposite material 215 can comprise a polymer material (also referred to as a polymer network/matrix) 225 and a plurality of conductive nanoparticles 220 dispersed in the polymer material. The plurality of conductive nanoparticles 220 can be many different types of conductive nanoparticles known in the art, including, but not limited to, gold nanoparticles, silver nanoparticles, carbon nanoparticles, the like, and combinations thereof. As shown in FIG. 8, the plurality of conductive nanoparticles 220 can be homogeneously dispersed in the polymer material 225.

The plurality of nanoparticles can have many different sizes, in accordance with various embodiments of the present disclosure. In some embodiments, the plurality of nanoparticles can have an average particle size of less than 10 nm (e.g., 1-10 nm), less than 5 nm (e.g., 1-5 nm or 4-5 nm), or other sizes.

In any of the embodiments disclosed herein, the nanocomposite material 215 can be in the form of a nanocomposite film. The nanocomposite material 215 can be in electrical communication with the first 205 and second 210 electrodes, such that electrical connections to the first 205 and second 210 electrodes can allow for measurement of an electrical property (e.g., impedance) of the nanocomposite material 215.

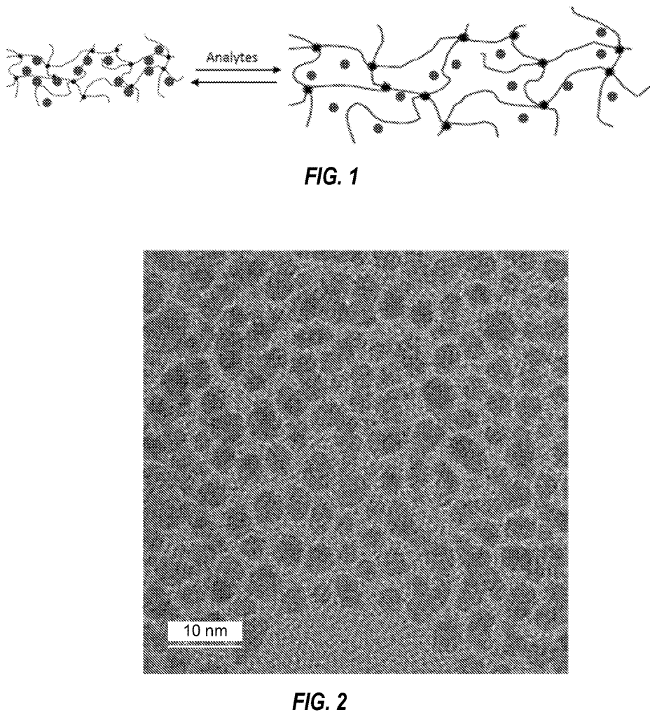

The polymer material 225 can be many different polymers or combinations of polymers known in the art. In some embodiments, the polymer material 225 can be selected based on the particular analyte to be detected. Exposure of the analyte to the nanocomposite material 215 can cause a swelling of the polymer material 225, which alters a spacing of the plurality of conductive nanoparticles 220 within the polymer material 225 (as shown in FIG. 1). The change in interparticle distance within the polymer material 225 due to exposure of the analyte to the polymer material 225 can alter the impedance (e.g., resistance and/or capacitance) across the nanocomposite material 215 between the first 205 and second 210 electrodes. A measurement of this change of impedance can be indicative of the presence/level of the analyte.

As discussed in the examples below, it can be desirable to closely match the Hansen solubility parameters of the polymer material 225 and the analyte, which can lead to increased sensitivity of the system to detect the analyte. Accordingly, in some embodiments, the polymer material 225 can be selected such that a difference between the Hansen solubility parameter of the polymer material and the analyte is less than 1, less than 0.75, less than 0.5, less than 0.25, or less than 0.1.

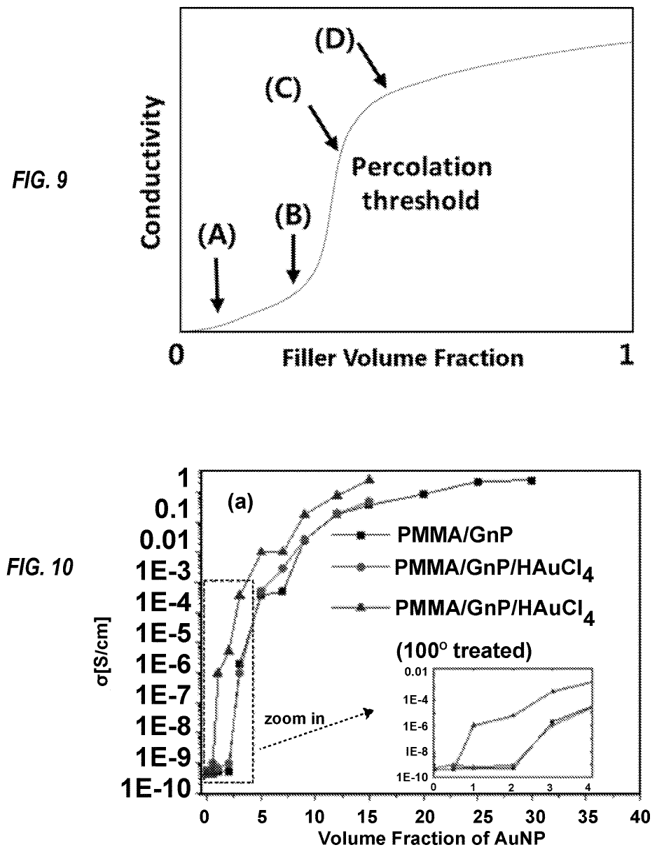

FIG. 9 provides a graph that exemplifies the relationship between nanocomposite conductivity and filler volume fraction of nanoparticles contained within a polymer that forms a nanocomposite film. In particular, in the first stage (A), the conductivity of the nanocomposite film is low as a small number of nanoparticles are isolated inside the polymer. The conductivity of the composite material or film is almost the same as that of the polymer. In the second stage (B), clusters of nanoparticles are formed with more filler nanoparticles inside the polymer. In this state, tunneling can occur between adjacent nanoparticles, resulting in a gradual increase in the conductivity of the composite material or film. In the third phase (C), a complete electrical path is formed between the nanoparticles as the number of nanoparticles approaches percolation threshold. The conductivity of composite materials or films at this stage increases rapidly. In the final phase (D), as more filler nanoparticles are added to the polymer, the conductivity of the composite material or film gradually increases. The most sensitive sensing response for the nanocomposite film can be obtained when the nanoparticle volume fraction is between stages (B) and (C).

FIG. 10 provides a graph illustrating the relationship between conductivity of a nanocomposite or film containing gold nanoparticles (GnP) with and without chloroauric acid (HAuCl4) in an acrylate polymer (PMMA) based on the volume fraction of gold nanoparticles present in the film. As known in the art, the conductivity σ (S/cm) of a nanocomposite or film is related to the resistance R based on the following formula:

R=L/(σA)

where L is the distance or gap G between electrodes and A is the cross-sectional area of the coated nanocomposite film on the electrode. In some embodiments of the present disclosure, the targeted conductivity for the nanocomposite film can be in the range from about 1E-8 S/cm to about 1E-3 S/cm, which corresponds to a resistance value of from about 100 kiloohms to 1000 about Megaohms. However, due to issues with the accuracy of measuring high resistances (e.g., those above 10 Megaohms) from the leakage current, in some embodiments of the present disclosure, the targeted resistance of the can range from about 100 kiloohms to about 10 Megaohms.

The system can provide for improved sensitivity to various analytes over conventional sensors. In some embodiments, the system can be configured to detect the presence of the analyte when the analyte is exposed to the polymer material at a concentration level of 100 parts per billion (ppb), 75 ppb, 50 ppb, 25 ppb, or lower.

The system can be configured to detect many different analytes. In some embodiments, the analyte can be a volatile organic compound (VOC). For example, the VOC can be a VOC produced by a microorganism. Therefore, the sensor can be used to detect the presence of the bacteria (e.g., bacteria resulting from improper cleaning of an autoclave) by detecting the presence of VOCs emitted by the bacteria. The VOCs can be many different VOCs, including, but not limited to, cyclododecanol, benzoic acid, 4-ethoxy-, ethyl ester, 3,4,4-trimethyl-3-pentanol, the like, and combinations thereof.

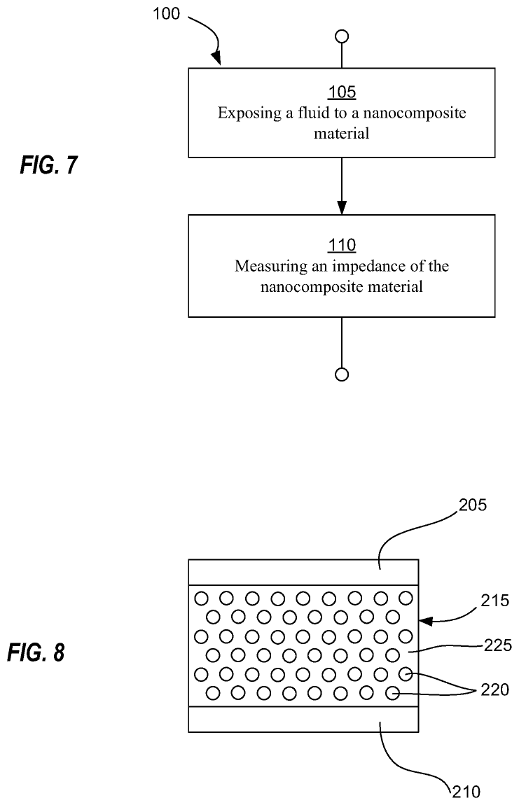

In addition to sensor systems, the present disclosure also provides methods of detecting whether an analyte is present in a fluid (e.g., the headspace vapor of a liquid sample). As shown in FIG. 7, an exemplary method 100 can comprise: exposing the fluid to a nanocomposite material 105; and measuring an impedance of the nanocomposite material 110. The impedance can be indicative of whether the analyte is present in the fluid.

As discussed above, exposing the fluid to the nanocomposite material 105 can cause the nanocomposite material to swell, altering a spacing of the plurality of conductive nanoparticles within the polymer material. This changing of spacing of the nanoparticles can alter an impedance across the nanocomposite material, which can be measured to detect whether and to what extend the analyte is present in the fluid.

### EXAMPLES

The following examples are provided to illustrate various embodiments of the present disclosure. These examples, however, should not be construed as limiting the scope of the present disclosure of the claims appended hereto.

### Sensing Film Design

Conductive nanoparticles dispersed in a VOC-responsive polymer network was used as the sensing film. Swelling of the polymer upon exposure to a gas/vapor increases the interparticle distances, and results in an increased resistance of the film. The sensing mechanism can be found in FIG. 1.

VOC responsive polymer film was selected based on the Flory-Huggins equation, as shown in equation 1:

$${χ}_{12} = \frac{V}{R T} {( {δ}_{TPol} - {δ}_{T S o l} )}^{2} \qquad ( Equation 1 )$$

where χ₁₂ is the interaction parameter, V is the volume of 1 mole of polymer segments, and δ_{TPol} and δ_{TSol} are total solubility parameters if polymer and solvent respectively, R is the gas constant (8.314 J/(mol·K)), T is the temperature (in K). The highest interaction between polymer chains and the organic molecules can be achieved when the χ12, the Flory-Huggins parameter, is close to zero. Therefore, matching solubility parameters between the polymer and targeted VOC can be desired for increased detection sensitivity. Three intermolecular interactions are included in the Hansen solubility calculation as shown in Equation 2:

$${δ}_{T}^{2} = {δ}_{d}^{2} + {δ}_{p}^{2} + {δ}_{h}^{2} \qquad ( Equation 2 )$$

where, δd is dispersion component parameter, δp is polar component parameter, and δh is hydrogen bonding component parameter. By choosing a suitable polymer and exploiting the different X-values between the polymer and the targeted VOC, selective detection can be achieved.

To convert the polymer/VOC interaction into a measurable signal, conductive nanoparticles were added into the polymer network. Gold nanoparticles were selected due to their tunable chemistries that permits controlled and facile variations to be made in the size of the gold core and thickness of the ligand shell. These clusters can be very stable in the solid state and easily redisperse in nonpolar organic solvents. The conductivity of the gold nanoparticles can be varied by adjusting the gold to alkanethiol stoichiometry and the length of the ligand.

The TEM (transmission electron microscopy) images of the synthesized gold nanoparticles can be found in FIG. 2. Based on the TEM measurements, the particle size was around 4 to 5 nm.

Preliminary Sensing with Gold Nanoparticle/Polymer Composite

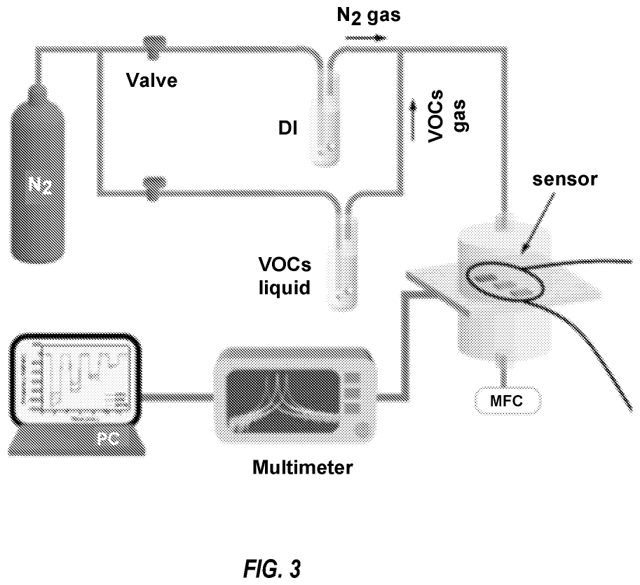

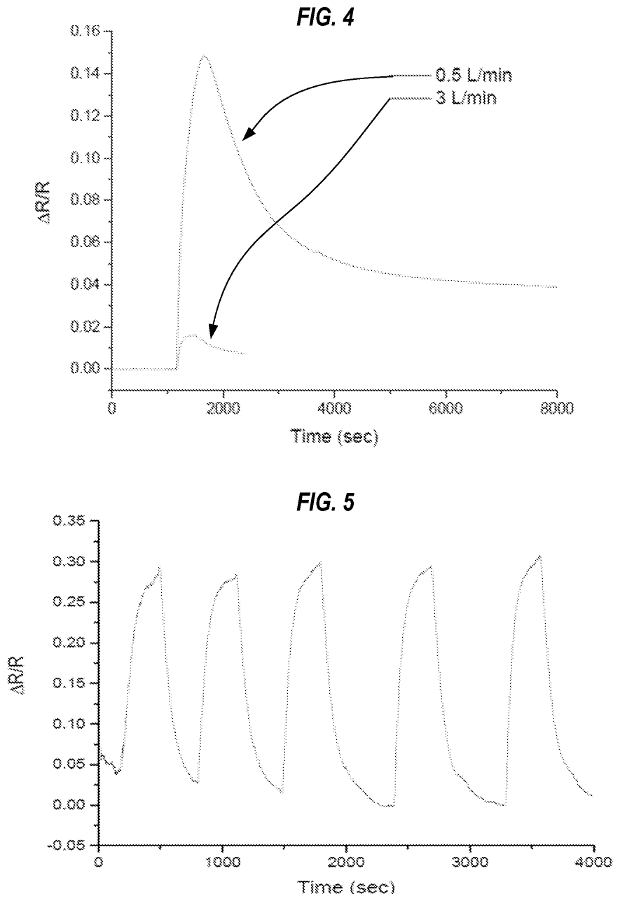

The nanoparticle/polymer mixture can be applied on the sensing electrodes via drop-casting, aerosol-jet printing, ink-jet printing or similar processes. The experimental setup for VOC sensing can be found in FIG. 3, where a flow of nitrogen passed through a VOC containing water sample held in a temperature controlled centrifuge tube. The headspace sample was then delivered to the flow cell for the impedance (e.g., resistance and/or capacitance) measurement. The deionized water in the second centrifuge tube was used as a reference for the baseline correction. The sensing data for the 300 ppm of 2-decanone prepared in deionized water can be found in FIG. 4. An increased response was obtained with a faster flow of nitrogen.

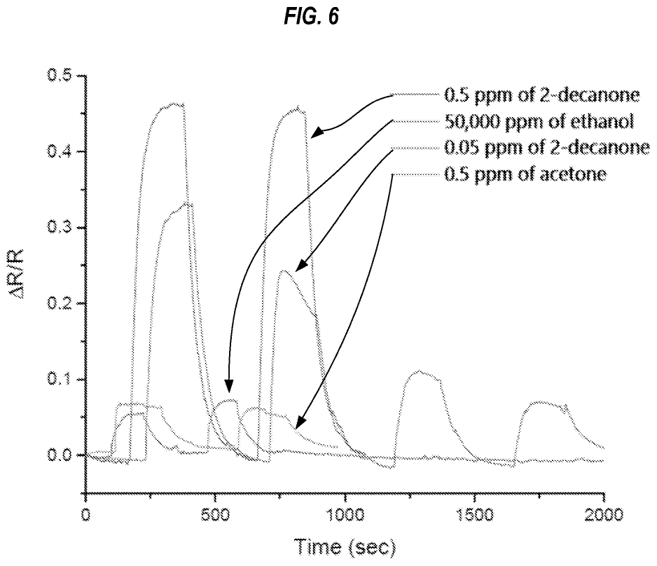

The sensing responses can be optimized by adjusting the doping level of the gold nanoparticles inside the polymer solution. The sensing responses are fast and reproducible. Five consecutive VOC detections can be found in FIG. 5. The selective of the sensing film was also examined toward 50,000 ppm of ethanol and 0.5 ppm of acetone prepared in DI. Much greater responses were obtained to 2-decanone compared to those obtained from ethanol and acetone, as shown in FIG. 6.

It is to be understood that the embodiments and claims disclosed herein are not limited in their application to the details of construction and arrangement of the components set forth in the description and illustrated in the drawings. Rather, the description and the drawings provide examples of the embodiments envisioned. The embodiments and claims disclosed herein are further capable of other embodiments and of being practiced and carried out in various ways. Also, it is to be understood that the phraseology and terminology employed herein are for the purposes of description and should not be regarded as limiting the claims.

Accordingly, those skilled in the art will appreciate that the conception upon which the application and claims are based may be readily utilized as a basis for the design of other structures, methods, and systems for carrying out the several purposes of the embodiments and claims presented in this application. It is important, therefore, that the claims be regarded as including such equivalent constructions.

Furthermore, the purpose of the foregoing Abstract is to enable the United States Patent and Trademark Office and the public generally, and especially including the practitioners in the art who are not familiar with patent and legal terms or phraseology, to determine quickly from a cursory inspection the nature and essence of the technical disclosure of the application. The Abstract is neither intended to define the claims of the application, nor is it intended to be limiting to the scope of the claims in any way.

1. A system comprising:
a first electrode;
a second electrode; and
a nanocomposite material comprising a polymer material and conductive nanoparticles dispersed in the polymer material;
wherein the nanocomposite material is in electrical communication with the first and second electrodes.

2. The system of claim 1, wherein the system is configured to detect the presence of a volatile organic compound (VOC) selected from the group consisting of cyclododecanol, benzoic acid, 4-ethoxy-, ethyl ester, 3,4,4-trimethyl-3-pentanol, and combinations thereof such that exposure of the VOC to the nanocomposite material alters an impedance between the first and second electrodes.

3. The system of claim 1, wherein:
the system is configured to detect the presence of an analyte such that exposure of the analyte to the nanocomposite material causes the nanocomposite material to swell, altering a spacing of the conductive nanoparticles within the polymer material; and
at least one of:
the conductive nanoparticles are gold nanoparticles; or
the conductive nanoparticles have an average particle size of less than 10 nm.

4. A system for detecting the presence of an analyte comprising:
a first electrode;
a second electrode; and
a nanocomposite material comprising a polymer material and conductive nanoparticles dispersed in the polymer material;
wherein:
the nanocomposite material is in electrical communication with the first and second electrodes;
the polymer material has a first set of Hansen solubility parameters;
the analyte has a second set of Hansen solubility parameters; and
a difference between the first set of Hansen solubility parameters and the second set of Hansen solubility parameters results in an interaction parameter χ₁₂ in the Flory-Huggins equation of less than 1; and
wherein the system is configured such that exposure of the analyte to the nanocomposite material at least one of:
alters an impedance between the first and second electrodes; or
causes the nanocomposite material to swell, altering a spacing of the conductive nanoparticles within the polymer material.

5. The system of claim 4, wherein the difference between the first set of Hansen solubility parameters and the second set of Hansen solubility parameters results in the interaction parameter χ₁₂ in the Flory-Huggins equation of less than 0.75.

6. The system of claim 4, wherein the difference between the first set of Hansen solubility parameters and the second set of Hansen solubility parameters results in the interaction parameter χ₁₂ in the Flory-Huggins equation of less than 0.5.

7. The system of claim 4, wherein the system is further configured to detect the presence of the analyte when the analyte is exposed to the polymer material at a concentration level of 50 parts per billion (ppb).

8. The system of claim 4, wherein the analyte is a volatile organic compound (VOC).

9. The system of claim 8, wherein the VOC is selected from the group consisting of cyclododecanol, benzoic acid, 4-ethoxy-, ethyl ester, 3,4,4-trimethyl-3-pentanol, and combinations thereof.

10. The system of claim 4, wherein the conductive nanoparticles are selected from the group consisting of gold nanoparticles, silver nanoparticles, and carbon nanoparticles.

11. The system of claim 4, wherein the conductive nanoparticles are gold nanoparticles.

12. The system of claim 4, wherein at least one of:
the conductive nanoparticles are homogeneously dispersed in the polymer material; or
the nanocomposite material is in the form of a nanocomposite film.

13.-14. (canceled)

15. The system of claim 4, wherein the conductive nanoparticles have an average particle size of less than 5 nm.

16. A method of using the system of claim 1 to detect whether an analyte is present in a fluid comprising:
exposing the fluid to the nanocomposite material; and
measuring with the first and second electrodes, after exposing the fluid to the nanocomposite material, an impedance of the nanocomposite material;
wherein the impedance is indicative of whether the analyte is present in the fluid.

17. The method of claim 16, wherein exposing the fluid to the nanocomposite material causes the nanocomposite material to swell, altering a spacing of the conductive nanoparticles within the polymer material.

18. The method of claim 17, wherein:
the polymer material has a first set of Hansen solubility parameters;
the analyte has a second set of Hansen solubility parameters and
a difference between the first set of Hansen solubility parameters and the second set of Hansen solubility parameters results in an interaction parameter χ₁₂ in the Flory-Huggins equation of less than 1.

19. (canceled)

20. The method of claim 18, wherein the difference between the first set of Hansen solubility parameters and the second set of Hansen solubility parameters results in the interaction parameter χ₁₂ in the Flory-Huggins equation of less than 0.5.

21. The method of claim 18, wherein the method is capable of detecting the presence of the analyte when the analyte is present in the fluid at a concentration level of 50 parts per billion (ppb).

22. (canceled)

23. The method of claim 18, wherein the analyte is a volatile organic compound (VOC) selected from the group consisting of cyclododecanol, benzoic acid, 4-ethoxy-, ethyl ester, 3,4,4-trimethyl-3-pentanol, and combinations thereof.

24. (canceled)

25. The method of claim 18, wherein at least one of:
the conductive nanoparticles are gold nanoparticles;
the conductive nanoparticles are silver nanoparticles;
the conductive nanoparticles are carbon nanoparticles;
the conductive nanoparticles are homogeneously dispersed in the polymer material;
the nanocomposite material is in the form of a nanocomposite film;
the conductive nanoparticles have an average particle size of less than 10 nm; or
the conductive nanoparticles have an average particle size of less than 5 nm.

26.-29. (canceled)
